(19) United States
(12) Patent Application Publication
(10) Pub. No.: US 2021/0202042 A1
(43) Pub. Date: Jul. 1, 2021

(54) INFORMATION CLASSIFICATION PROCESSING METHOD OF CARBONATE RESERVOIR AND INFORMATION DATA PROCESSING TERMINAL

(71) Applicant: Chengdu University of Technology, Chengdu (CN)

(72) Inventors: Meiyan FU, Chengdu (CN); Hucheng DENG, Chengdu (CN); Wen ZHOU, Chengdu (CN); Xiaobo GUO, Chengdu (CN); Tao LU, Chengdu (CN); Chenyang ZHAO, Chengdu (CN); Liang ZHAO, Chengdu (CN); Pei CHEN, Chengdu (CN)

(21) Appl. No.: 17/038,362

(22) Filed: Sep. 30, 2020

(30) Foreign Application Priority Data
Dec. 31, 2019 (CN) 201911421446.0

(51) Int. Cl. G16B 40/00; G01N 33/24; E21B 49/02; E21B 49/00

(52) U.S. Cl. CPC G16B 40/00; E21B 49/005; E21B 49/02; G01N 33/24

(57) ABSTRACT

An information classification processing method of a carbonate reservoir and an information data processing terminal. The method includes: determining rock types; determining reservoir types on the basis of different rock types; and in accordance with the determined reservoir and rock types, performing porosity and permeability intersection by utilizing measured data; fitting a curve to obtain a porosity-permeability relation formula; and calculating permeability by utilizing the formula. According to the present invention, complex carbonate reservoirs in the Middle East can be classified, and porosity-permeability relations are respectively established, thereby increasing interpretation accuracy of permeability. According to the present invention, the reservoir types and the rock types can be rapidly and systematically classified, and clear porosity-permeability relations are obtained, so that interpretation of the permeability in oil reservoir exploitation is more accurate. The system has been applied to Halfaya Oilfield in Iraq of Middle East.

### CROSS-REFERENCE TO RELATED APPLICATIONS

This application claims the benefit of priority from Chinese Patent Application No. 201911421446.0, filed on Dec. 31, 2019. The content of the aforementioned applications, including any intervening amendments thereto, is incorporated herein by reference in its entirety.

### TECHNICAL FIELD

The present invention belongs to the technical field of information processing and particularly relates to an information classification processing method of a carbonate reservoir and an information data processing terminal.

### BACKGROUND OF THE PRESENT INVENTION

At present, in the closest prior art, there are two types of carbonate reservoirs discovered in current global oil-gas exploration, i.e., pore-type or pore-fracture type reservoirs, and fracture-vug type reservoirs. There are two categories in the pore-type reservoirs, such as limestone and dolostone. The carbonate reservoirs in the Middle East are mainly pore-type limestone reservoirs. However, under the control of sedimentary facies and near-surface diagenesis, components and types of the limestone are very complex; the relationship between porosity and permeability in the reservoirs is poor; and permeability interpretation is very difficult, thereby limiting the identification for the reservoirs and formulation of oil and gas development strategy.

To sum up, the prior art has the following problems: under the control of the sedimentary facies and the near-surface diagenesis, the components and the types of the limestone are very complex; the relationship between porosity and permeability of the reservoirs is poor; and the permeability interpretation is very difficult, thereby limiting the identification for the reservoirs and formulation of the oil and gas development strategy.

### SUMMARY OF THE PRESENT INVENTION

With respect to the problems in the prior art, the present invention provides an information classification processing method of a carbonate reservoir and an information data processing terminal.

The present invention is realized as follows: the information classification processing method of the carbonate reservoir is provided; and the information classification processing method of the carbonate reservoir includes the following steps:

step 1: determining rock types;

step 2: determining reservoir types on the basis of different rock types;

step 3: in accordance with relative content of a lime mud matrix determined in each rock sample, low-energy particles and high-energy particles, determining corresponding reservoir and rock types in combination with pore types; after analyzing the reservoir and rock types of all the rock samples, performing porosity and permeability intersection on measured porosity and permeability data corresponding to each reservoir type and each rock type, and performing regression fitting to obtain permeability calculation formulas taking the porosity as a function, wherein totally 6 formulas of reservoir rocks are formed (marlstone is not a reservoir rock); and by taking logging porosity as a function, calculating corresponding permeability of reservoir and rock types by utilizing the above permeability formulas.

Further, the step 1 of determining rock types includes:

(1) observing and identifying the rock thin sections for components of limestone in the Middle East;

(2) counting relative content of the lime mud matrix, low-energy particles and high-energy particles, wherein the low-energy particles refer to green algae, bivalve and Denthic foraminifera that deposit in an environment having weak energy; and the high-energy particles refer to shellfishes, rudistids, Echinodermata, Bryozoans, stromatoporoids and corals that deposit in an environment having strong energy;

(3) normalizing the relative content of the above three components by utilizing a layout, and then performing cultellation; and determining corresponding rock types through cultellation.

Further, in the step 2, the seven reservoir types are determined on the basis of different rock types:

(1) marlstone is not a reservoir, and content of the lime mud matrix is greater than 90%;

(2) wackestone is a poor reservoir and has particle content of 10-50%; the rock structure is of a matrix support structure; pores are mainly intercrystalline pores; a small amount of moldic pores are developed; the distribution of pore throat radius has a bimodal pattern; and a small pore throat is dominant;

(3) low-energy particle limestone is a poor or worse reservoir and has particle content of more than 50%; pores are mainly organism cavity pores and moldic pores; and the limestone is of a large-pore and fine-throat type;

(4) mixed particle limestone II is a poor reservoir and has particle content of more than 50%; pores are mainly intercrystalline pores and body cavity pores; a few biological moldic pores are developed; the distribution of the pore throat radius has a bimodal pattern; and through comparison, the two pore throat types are not obvious dominant types;

(5) mixed particle limestone I is a better reservoir and has particle content of more than 50%; pores are mainly inter-particle pores; moldic pores and organism cavity pores are developed; the pore type has duality; the distribution of the pore throat radius has a bimodal pattern; a large pore throat type is dominant; and the throat is mainly of a necking type;

(6) high-energy particle limestone II is a better reservoir and has particle content of more than 50%; however, a certain amount of lime mud exists among the particles; a combination of inter-particle pores, inter-particle dissolved pores and intercrystalline pores is formed; the pores are mainly the intercrystalline pores; the throat is thick; permeability is high; the distribution of the pore throat radius does not have an obvious bimodal pattern; and a large pore throat type is dominant;

(7) high-energy particle limestone I is a good reservoir and has particle content of more than 75%; almost no lime mud matrix exists among the particles; the intercrystalline pores are dominant in the pore types; a small amount of inter-particle dissolved pores may be formed; the pores are mainly the intercrystalline pores; the throat is thick; the permeability is high; in the distribution of the pore throat radius, the large pore throat type is dominant; and for the large pore thick throat, the throat is mainly of a pore necking type.

Another purpose of the present invention is to provide an information classification processing system of a carbonate reservoir for implementing the information classification processing method of the carbonate reservoir. The information classification processing system of the carbonate reservoir includes:

a rock type determining module for determining corresponding rock types;

a reservoir type determining module for determining reservoir types on the basis of different rock types;

a porosity-permeability relation determining module of different reservoir types for performing porosity and permeability intersection in accordance with the determined reservoir and rock types by utilizing measured data, and fitting a curve to obtain porosity-permeability relations so as to calculate the permeability.

Another purpose of the present invention is to provide an information data processing terminal for realizing the information classification processing method of the carbonate reservoir.

Another purpose of the present invention is to provide a computer readable storage medium including instructions. When the instructions are executed on a computer, the computer executes the above information classification processing method of the carbonate reservoir.

In conclusion, the present invention has the advantages and positive effects as follows: in the present invention, complex carbonate reservoirs in the Middle East can be classified, and the porosity-permeability relations are respectively established, thereby increasing the interpretation accuracy of the permeability. According to the present invention, the reservoir and rock types can be rapidly and systematically classified, and clear porosity-permeability relations are obtained, so that the interpretation of the permeability in oil reservoir exploitation is more accurate. The system has been applied to Halfaya Oilfield in Iraq of the Middle East.

### BRIEF DESCRIPTION OF THE DRAWINGS

FIG. 1 is a structural schematic diagram of an information classification processing system of a carbonate reservoir provided in embodiments of the present invention;

In FIG. 1, 1: rock type determining module; 2: reservoir type determining module; 3: porosity-permeability relation determining module of different reservoir types;

FIG. 2 is a flow chart of an information classification processing method of a carbonate reservoir provided in embodiments of the present invention; and

FIG. 3 is a layout diagram provided in embodiments of the present invention.

### DETAILED DESCRIPTION OF THE PRESENT INVENTION

To make the purposes, technical solutions and advantages of the present invention more clear, the present invention will be further described below in detail in combination with embodiments. It should be understood that, specific embodiments described herein are merely used for illustrating the present invention, rather than limiting the present invention.

With respect to the problems in the prior art, the present invention provides an information classification processing method of a carbonate reservoir and an information data processing terminal. The present invention is described below in detail in combination with drawings.

As shown in FIG. 1, an information classification processing system of the carbonate reservoir provided by embodiments of the present invention includes:

a rock type determining module 1 for determining corresponding rock types;

a reservoir type determining module 2 for determining reservoir types on the basis of different rock types;

a porosity-permeability relation determining module 3 of different reservoir types for performing porosity and permeability intersection in accordance with the determined reservoir and rock types by utilizing measured data, and fitting a curve to obtain porosity-permeability relations so as to calculate permeability.

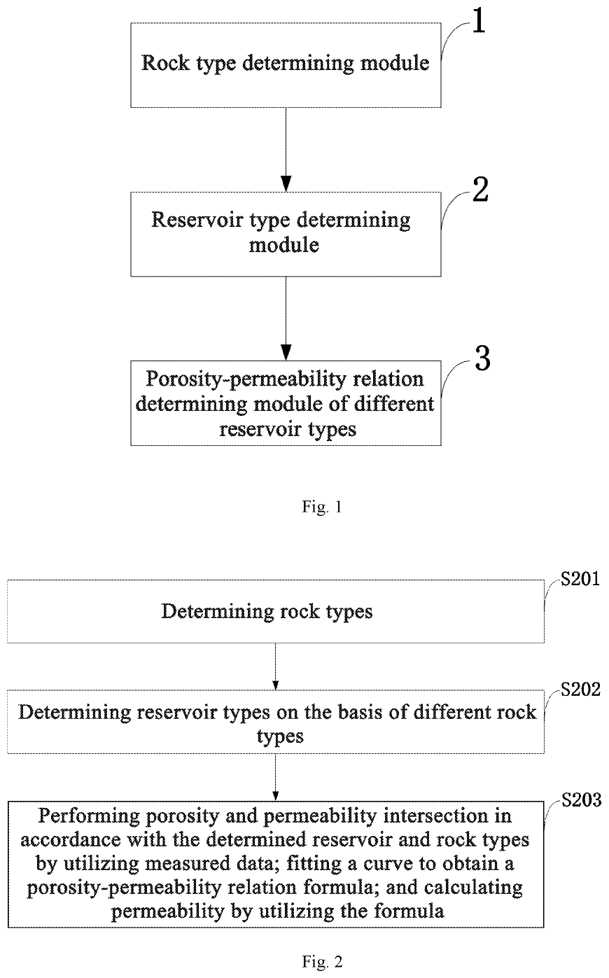

As shown in FIG. 2, an information classification processing method of a carbonate reservoir provided by embodiments of the present invention includes the following steps:

S201: determining rock types;

S202: determining reservoir types on the basis of different rock types;

S203: performing porosity and permeability intersection in accordance with the determined reservoir and rock types by utilizing measured data; fitting a curve to obtain porosity-permeability relation formulas; and calculating the permeability by utilizing the formulas.

The technical solutions of the present invention are further described below in combination with the drawings.

The information classification processing method of the carbonate reservoir provided by embodiments of the present invention specifically includes the following steps:

Step 1: determining rock types:

1. observing and identifying rock slices for components of limestone in the Middle East;

2. counting relative content of lime mud matrixes, low-energy particles and high-energy particles, wherein the low-energy particles refer to green algae, bivalve and Denthic foraminifera that deposit in an environment having weak energy; and the high-energy particles refer to shellfishes, rudistids, Echinodermata, Bryozoans, stromatoporoids and corals that deposit in an environment having strong energy;

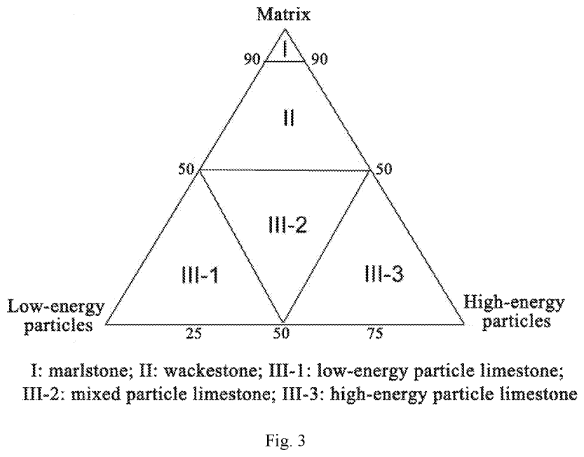

3. normalizing the relative content of the above three components by utilizing a layout below (as shown in FIG. 3), and then performing cultellation; and determining corresponding rock types through cultellation.

Step 2: determining the reservoir types:

The reservoir types are determined on the basis of different rock types, and have 7 types:

1. marlstone is not a reservoir, and content of the lime mud matrix is greater than 90%;

2. wackestone is a poor reservoir and has particle content of 10-50%; the rock structure is of a matrix support structure; pores are mainly intercrystalline pores; a small amount of moldic pores are developed; the distribution of pore throat radius has a bimodal pattern; and a small pore throat is dominant;

3. low-energy particle limestone is a poor or worse reservoir and has particle content of more than 50%; pores are mainly organism cavity pores and moldic pores; and the limestone is of a large-pore fine-throat type;

4. mixed particle limestone II is a poor reservoir and has particle content of more than 50%; pores are mainly intercrystalline pores and body cavity pores; a few biological moldic pores are developed; the distribution of the pore throat radius has a bimodal pattern; and through comparison, the two pore throat types are not obvious dominant types;

5. mixed particle limestone I is a better reservoir and has particle content of more than 50%; pores are mainly inter-particle pores; mold pores and organism cavity pores are developed; the pore type has duality; the distribution of the pore throat radius has a bimodal pattern; a large pore throat type is dominant; and the throat is mainly of a necking type;

6. high-energy particle limestone II is a better reservoir and has particle content of more than 50%; however, a certain amount of lime mud exists among the particles; a combination of inter-particle pores, inter-particle dissolved pores and intercrystalline pores is formed; the pores are mainly the intercrystalline pores; the throat is thick; permeability is high; the distribution of the pore throat radius does not have an obvious bimodal pattern; and a large pore throat type is dominant;

7. high-energy particle limestone I is a good reservoir and has particle content of more than 75%; almost no lime mud matrix exists among the particles; the intercrystalline pores are dominant in the pore types; a small amount of inter-particle dissolved pores may be formed; the pores are mainly the intercrystalline pores; the throat is thick; the permeability is high; in the distribution of the pore throat radius, the large pore throat type is dominant; and for the large pore thick throat, the throat is mainly of a pore necking type.

Step 3: obtaining porosity-permeability relations of different reservoir types: performing porosity and permeability intersection in accordance with the determined reservoir and rock types by utilizing measured data, and fitting a curve to obtain a porosity-permeability relation formula, wherein the permeability can be calculated by utilizing the formula; determining corresponding reservoir and rock types in combination with pore types according to the relative content of the lime mud matrix of each rock sample, the low-energy particles and the high-energy particles; after analyzing reservoir and rock types of all the rock samples, performing porosity and permeability intersection on measured porosity and permeability data corresponding to each reservoir type and each rock type, and performing regression fitting to obtain permeability calculation formulas taking the porosity as a function, wherein totally 6 formulas of reservoir rocks are formed (marlstone is not a reservoir rock); and by taking logging porosity as a function, calculating the corresponding permeability of reservoir and rock types by utilizing the above permeability formulas.

The present invention is applied to two oilfields in Middle and South of Iraq of Middle East, and is recognized by Overseas Research Center of China National Petroleum Corporation. By researching cretaceous carbonate reservoirs, it is considered that, the development of the reservoirs in Iraq is mainly controlled by a deposition process, and basic structures of the reservoirs are influenced by particle types and the content of the lime mud. Moreover, due to near-surface diagenesis, carbonate undergoes karst erosion; thus, the depositional texture is transformed partially, not strongly. Based on the above geological researches, it is determined that, the reservoir and rock types in Iraq can be classified into 7 categories; the porosity-permeability relations corresponding to the reservoir and rock types of 6 types of reservoir rocks are obtained; and the permeability calculation formulas taking the porosity as the function are established. Conformity of the permeability calculated by utilizing the formula and measured permeability is higher than that in previous researches, which shows the applicability and inventiveness of the present invention.

It should be noted that, the embodiments of the present invention may be realized by hardware, software or a combination of software and hardware. The hardware part may be realized by utilizing a special logic; and the software part may be stored in a memory and executed by an appropriate instruction execution system, such as a microprocessor or special design hardware. Those ordinary skilled in the art may understand that, the above equipment and method may be realized by using computer executable instructions and/or control codes included in the processor. For example, these codes are provided on carrier media such as disk, CD or DVD-ROM, a programmable memory such as read-only memory (firmware) or a data carrier such as an optical or electronic signal carrier. The equipment and modules thereof in the present invention can be realized by super-large-scale integrated circuit or gate array, semiconductors such as logic chips and transistors, or hardware circuits of programmable hardware equipment such as field-programmable gate arrays and programmable logic devices, can also be realized by software executed by various types of processors, and can also be realized by a combination of the above hardware circuits and software, e.g., the firmware.

The above only describes preferred embodiments of the present invention, not intended to limit the present invention. Any modification, equivalent replacement and improvement made within the spirit and principle of the present invention shall be included in the protection scope of the present invention.

We claim:

1. An information classification processing method of a carbonate reservoir, wherein the information classification processing method of the carbonate reservoir comprises the following steps:
step 1: determining rock types;
step 2: determining reservoir types on the basis of different rock types;
step 3: performing porosity and permeability intersection in accordance with the determined reservoir and rock types by utilizing measured data; fitting a curve to obtain porosity-permeability relation formulas; and calculating the permeability by utilizing the formulas.

2. The information classification processing method of the carbonate reservoir according to claim 1, wherein the step 1 of determining rock types comprises:
(1) observing and identifying rock slices for components of limestone in the Middle East;
(2) counting relative content of lime mud matrixes, low-energy particles and high-energy particles, wherein the low-energy particles refer to green algae, bivalve and Denthic foraminifera that deposit in an environment having weak energy; and the high-energy particles refer to shellfishes, rudistids, Echinodermata, Bryozoans, stromatoporoids and corals that deposit in an environment having strong energy;
(3) normalizing the relative content of the above three components by utilizing a layout, and then performing cultellation; and determining corresponding rock types through cultellation.

3. The information classification processing method of the carbonate reservoir according to claim 1, wherein in the step 2, the seven reservoir types are determined on the basis of different rock types:
(1) marlstone is not a reservoir, and content of the lime mud matrix is greater than 90%;
(2) wackestone is a poor reservoir and has particle content of 10-50%; the rock structure is of a matrix support structure; pores are mainly intercrystalline pores; a small amount of moldic pores are developed; the distribution of pore throat radius has a bimodal pattern; and a small pore throat is dominant;
(3) low-energy particle limestone is a poor or worse reservoir and has particle content of more than 50%; pores are mainly organism cavity pores and moldic pores;
and the limestone is of a large-pore fine-throat type;
(4) mixed particle limestone II is a poor reservoir and has particle content of more than 50%; pores are mainly intercrystalline pores and body cavity pores; a few biological moldic pores are developed; the distribution of the pore throat radius has a bimodal pattern; and through comparison, the two pore throat types are not obvious dominant types;
(5) mixed particle limestone I is a better reservoir and has particle content of more than 50%; pores are mainly inter-particle pores; moldic pores and organism cavity pores are developed; the pore type has duality; the distribution of the pore throat radius has a bimodal pattern; a large pore throat type is dominant; and the throat is mainly of a necking type;
(6) high-energy particle limestone II is a better reservoir and has particle content of more than 50%; however, a certain amount of lime mud exists among the particles;
a combination of inter-particle pores, inter-particle dissolved pores and intercrystalline pores is formed; the pores are mainly the intercrystalline pores; the throat is thick; permeability is high; the distribution of the pore throat radius does not have an obvious bimodal pattern; and a large pore throat type is dominant;
(7) high-energy particle limestone I is a good reservoir and has particle content of more than 75%; almost no lime mud matrix exists among the particles; the intercrystalline pores are dominant in the pore types; a small amount of inter-particle dissolved pores may be formed; the pores are mainly the intercrystalline pores; the throat is thick; the permeability is high; in the distribution of the pore throat radius, the large pore throat type is dominant; and for the large pore thick throat, the throat is mainly of a pore necking type.

4. The information classification processing method of the carbonate reservoir according to claim 1, wherein the step 3 comprises: in accordance with relative content of the lime mud matrix determined in each rock sample, low-energy particles and high-energy particles, determining corresponding reservoir and rock types in combination with pore types; after analyzing the reservoir and rock types of all the rock samples, performing porosity and permeability intersection on measured porosity and permeability data corresponding to each reservoir type and each rock type, and performing regression fitting to obtain permeability calculation formulas taking the porosity as a function, wherein totally 6 formulas of reservoir rocks are formed; and by taking logging porosity as a function, calculating corresponding permeability of reservoir and rock types by utilizing the above permeability formulas.

5. An information classification processing system of a carbonate reservoir for implementing the information classification processing method of the carbonate reservoir of claim 1, wherein the information classification processing system of the carbonate reservoir comprises:
a rock type determining module for determining corresponding rock types;
a reservoir type determining module for determining reservoir types on the basis of different rock types;
a porosity-permeability relation determining module of different reservoir types for performing porosity and permeability intersection in accordance with the determined reservoir and rock types by utilizing measured data, and fitting a curve to obtain porosity-permeability relations so as to calculate the permeability.

6. An information data processing terminal for realizing the information classification processing method of the carbonate reservoir of claim 1.

7. An information data processing terminal for realizing the information classification processing method of the carbonate reservoir of claim 2.

8. An information data processing terminal for realizing the information classification processing method of the carbonate reservoir of claim 3.

9. An information data processing terminal for realizing the information classification processing method of the carbonate reservoir of claim 4.

10. A computer readable storage medium, comprising instructions, wherein when the instructions are executed on a computer, the computer executes the information classification processing method of the carbonate reservoir of claim 1.

11. A computer readable storage medium, comprising instructions, wherein when the instructions are executed on a computer, the computer executes the information classification processing method of the carbonate reservoir of claim 2.

12. A computer readable storage medium, comprising instructions, wherein when the instructions are executed on a computer, the computer executes the information classification processing method of the carbonate reservoir of claim 3.

13. A computer readable storage medium, comprising instructions, wherein when the instructions are executed on a computer, the computer executes the information classification processing method of the carbonate reservoir of claim 4.
